(19) United States
(12) Patent Application Publication
(10) Pub. No.: US 2020/0129580 A1
(43) Pub. Date: Apr. 30, 2020

(54) Supplements and Methods for Glucose Control and Liver Support

(71) Applicants: Sarthak Matravadia, Oakville (CA); Raza Bashir, Oakville (CA); William Ha, Oakville (CA); Vaishali Joshi, Oakville (CA)

(72) Inventors: Sarthak Matravadia, Oakville (CA); Raza Bashir, Oakville (CA); William Ha, Oakville (CA); Vaishali Joshi, Oakville (CA)

(73) Assignee: Northern Innovations Holding Corp., Oakville (CA)

(21) Appl. No.: 16/113,914

(22) Filed: Aug. 27, 2018

(51) Int. Cl. A61K 36/906; A61K 36/71; A61K 36/738; A61P 3/08; A61P 3/04

(52) U.S. Cl. CPC A61K 36/906; A61K 36/71; A61K 36/738; A61K 2236/333; A61P 3/04; A61K 2236/331; A61P 3/08

(57) ABSTRACT

A nutritional supplement comprising extracts Nigella sativa, Kaempferia parviflora, and Rosa canina and a method for blood glucose control and liver protection is provided. The nutritional supplement and method are particularly suited for use in conjunction with methods of weight loss. The supplement and method may also be used in other methods and compositions where it is desirable to control blood glucose levels and provide liver protection.

### FIELD OF THE INVENTION

The present invention relates generally to nutritional supplement compositions. More specifically, the present invention relates to a supportive blend of ingredients for blood glucose control and liver health support, particularly in weight loss compositions and methods.

### BACKGROUND OF THE INVENTION

The liver is an important organ that carries out many functions including blood detoxification, protein production, and production of factors involved in digestion and metabolism. It both stores and produces glucose—the preferred energy source for most vertebrate cell types—depending on the need. Blood glucose levels typically increase with feeding and decrease with exercise and fasting and are tightly controlled and held within a narrow range mainly by two opposing hormones: insulin and glucagon. Many disease states are associated with high levels of blood glucose. Therefore it is often desirable to control, by lowering or blunting, increases in blood glucose levels and to protect liver health and function.

### SUMMARY OF THE INVENTION

A composition is provided comprising Nigella sativa extract, Kaempferia parviflora extract, and Rosa canina extract.

In an embodiment of the invention, there is provided a method for controlling blood glucose levels using a composition comprising Nigella sativa extract, Kaempferia parviflora extract, and Rosa canina extract.

In another embodiment of the invention, there is provided a method for liver protection using a composition comprising Nigella sativa extract, Kaempferia parviflora extract, and Rosa canina extract.

In another embodiment of the invention, there is provided a method for controlling blood glucose levels and for liver protection using a composition comprising Nigella sativa extract, Kaempferia parviflora extract, and Rosa canina extract.

In some aspects, a composition comprising Nigella sativa extract, Kaempferia parviflora extract, and Rosa canina extract is used in conjunction with methods for weight loss.

### BRIEF DESCRIPTION OF THE DRAWINGS

Embodiments will now be described, by way of example only, with reference to the attached Figures, wherein:

FIG. 1 shows an overview of the study.

FIG. 2 shows the food consumption of the mice throughout the study.

FIG. 3 show the body weight of the mice throughout the study.

FIG. 4 shows the final body weight of the mice on the final treatment day.

FIG. 5 shows the body composition data of mice on the final treatment day.

FIG. 6 shows the non-fasted blood glucose values at Days 0, 10, and 31 for each diet group during the treatment period.

FIG. 7 compares the non-fasted blood glucose values at Days 0, 10, and 31 across each diet group.

FIG. 8 compares the fasted blood glucose values on the final treatment day.

FIG. 9 shows the mean serum alanine aminotransferase (ALT) values on the final study day.

FIG. 10 shows the mean serum aspartate aminotransferase (AST) values on the final study day.

### DETAILED DESCRIPTION OF THE EMBODIMENTS

As used herein, “blood glucose control” and like phrases are understood to refer to effects resulting in general lowering of high blood glucose levels, particularly non-fasted blood glucose levels.

As used herein, “improving liver health”, “supporting liver health”, and “promoting liver health” and like phrases are understood to refer to lowering markers of liver damage or improving liver function. The enzymes aspartate aminotransferase (AST) and alanine aminotransferase (ALT) are commonly measured as clinical biomarkers of liver health, with increases in one or both indicative of liver damage. A lowering of one or both of AST and ALT is understood to relate to improving liver health and/or liver function.

Nigella sativa (also known as black caraway, black cumin, fennel flower, nigella, nutmeg flower, Roman coriander, and kalonji) is a flowering plant native to Asia. It is widely used throughout the world as a medicinal plant, particularly the seeds, for conditions such as asthma, hypertension, diabetes, and inflammation.

Kaempferia parviflora (also known as Thai black ginger, Thai ginseng, Black Turmeric, Black Galingale, and krachai dum) is an herbaceous plant native to Thailand mainly used as an aphrodisiac.

Rosa canina (also known as dog rose and rose hip) is a deciduous shrub native to Europe, Africa, and Asia. Traditional uses include arthritis, gallstones, gout, and colds.

The inventors believed that the combination of Nigella sativa, Kaempferia parviflora, and Rosa canina would have beneficial effects on obesity, aiding in weight loss, through likely synergistic mechanisms. The inventors conducted a study in mice. Surprisingly, the inventors found that the specific combination of Nigella sativa, Kaempferia parviflora, and Rosa canina tested failed to result in observed weight loss while showing reductions in non-fasted blood glucose levels and markers of liver damage.

For the inventive composition, clinically relevant daily doses (in humans) for each of the ingredients were chosen and commercially available extracts were used. For Nigella sativa, 900 mg of a powdered 100% water seed extract at a ratio of 12-16:1 was used. For Kaempferia parviflora, 100 mg of a powdered dried root ethanol/water extract at a ratio of 3-4:1 was used. For Rosa canina, 100 mg of an ethanol/water fruit extract was used (containing about 0.1% trans-tiliroside). The ratio of Nigella sativa:Kaempferia parviflora:Rosa canina was 9:1:1 for a total of 1.1 g of the inventive composition.

### Example 1: Mouse Study

For testing of the inventive composition, the determined human doses were converted to equivalent doses for testing in mice. The mouse dosage (converted from the human dose) used was 200 mg/kg body weight at the same 9:1:1 ratio of Nigella sativa:Kaempferia parviflora:Rosa canina.

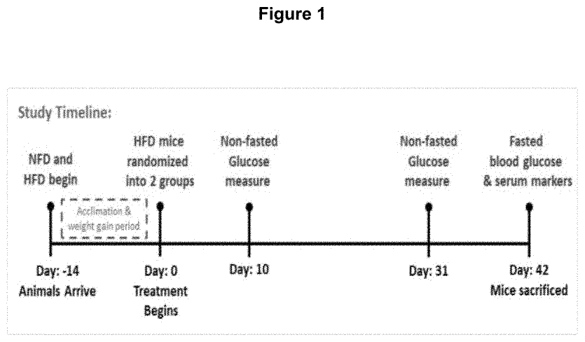

The mouse model used was the C57BL/6 DIO (Diet Induced Obesity) model. Three groups of 10 mice per group: NFD (normal fat diet), HFD (high fat diet), and HFD-Tx (high fat diet+200 mg/kg/day treatment). Mice were 16 weeks of age at the start of the study. The mice were acclimated for 14 days to the respective NFD or HFD diets after which time the HFD group was randomized and treatment was started. Non-fasted glucose was measured at 0, 10, and 31 days of treatment, while fasted blood glucose, and serum markers alanine aminotransferase (ALT) and aspartate aminotransferase (AST) were measured at 42 days of treatment, at which time all mice were sacrificed. The overall experimental setup is shown in FIG. 1.

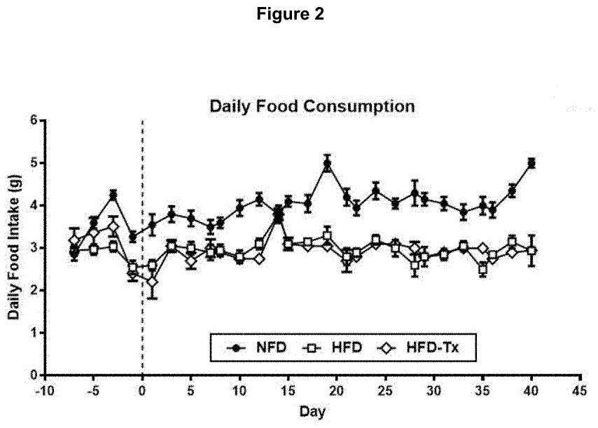

FIG. 2 shows the daily food consumption values of the mice during the entire experimental period. The average daily food intake of HFD and HFD-Tx (200 mg/kg/day) mice remained significantly lower than NFD mice throughout the study (*p<0.05). No significant differences in daily food intake were observed at any time point between HFD and HFD-Tx mice (p>0.05). Data are represented as means±SEM (standard error of the mean).

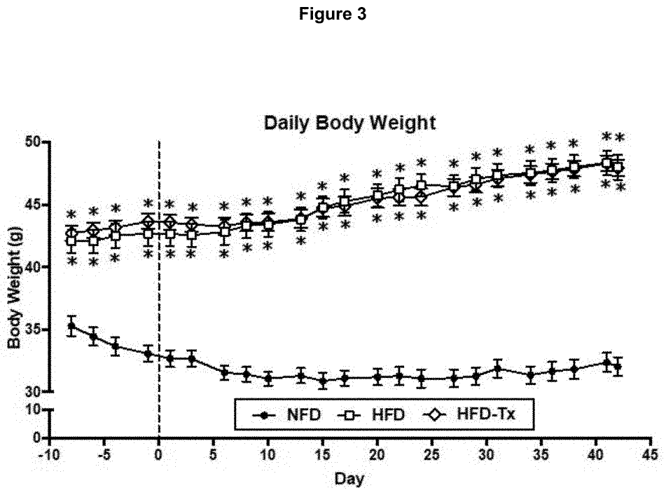

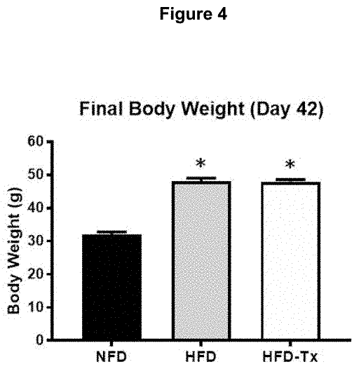

FIG. 3 shows the daily body weight of the mice during the entire experimental period. The average daily body weights of HFD and HFD-Tx (200 mg/kg/day) mice remained significantly higher than NFD mice throughout the study (*p<0.05). No significant differences in daily body weight were observed at any time point between HFD and HFD-Tx mice (p>0.05). Data are represented as means±SEM. FIG. 4 shows the mean body weight of each diet group on the final treatment day (day 42). The mean body weights of HFD and HFD-Tx (200 mg/kg/day) mice were significantly higher than NFD mice (*p<0.05) throughout the entire study period. No significant differences in mean body weight were found between HFD and HFD-Tx mice (p>0.05) at any time point. Data are represented as means±SEM.

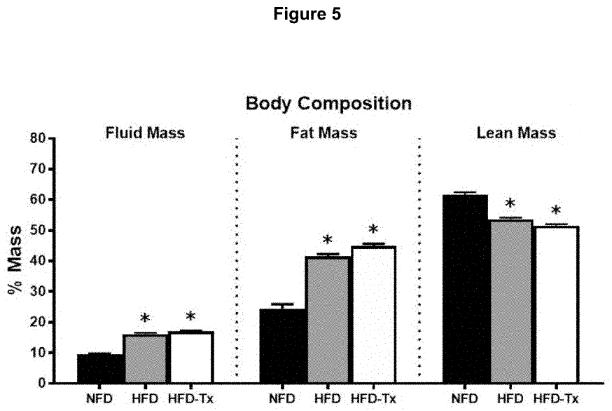

FIG. 5 shows the body composition data of mice on the final treatment day (day 42). The mean percentage of fluid and fat mass values of HFD and HFD-Tx (200 mg/kg/day) mice were significantly higher compared to NFD mice (*p<0.05). Whereas, percentage of lean mass was significantly lower in HFD and HFD-Tx mice compared to NFD mice (*p<0.05). No significantly differences were found between HFD and HFD-Tx mice (p>0.05) for any body composition measurements. Data are represented as means±SEM.

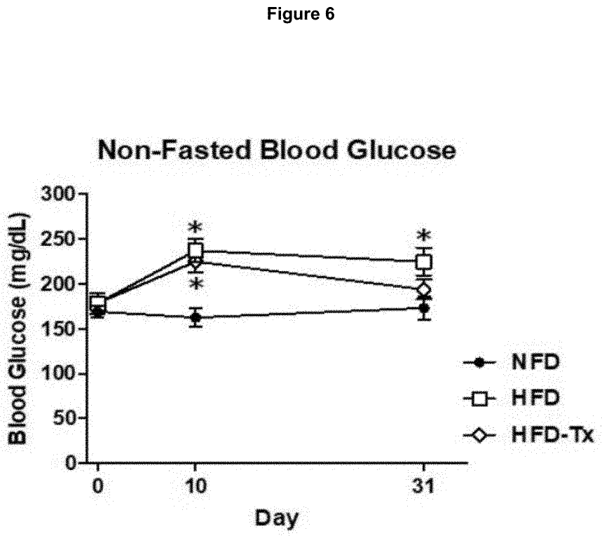

FIG. 6 shows the non-fasted blood glucose values at Days 0, 10, and 31 for each diet group during the treatment period. In NFD mice, non-fasted blood glucose values did not significantly vary across at any time point measured (p>0.05). In HFD mice, non-fasted blood glucose values were significantly elevated at Day 10 compared to Day 0 (*p<0.05), and remained elevated at Day 31 versus Day 0 (*p<0.05); values at Days 10 and 31 did not significantly vary. In HFD-Tx (200 mg/kg/day) mice, non-fasted blood glucose were significantly elevated at Day 10 compared to Day 0 (*p<0.05). However, at Day 31, non-fasted blood glucose values were significantly lower compared to Day 10 (*p<0.05), and were comparable to Day 0 values. Data are represented as means±SEM.

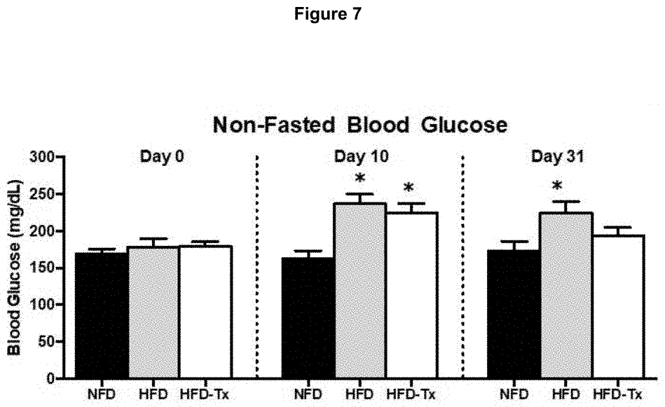

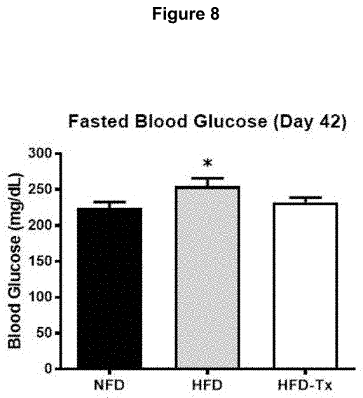

FIG. 7 compares the non-fasted blood glucose values at Days 0, 10, and 31 across each diet group. At Day 0, there were no significant differences between the diet groups. At Day 10, mean non-fasted blood glucose values of HFD and HFD-Tx (200 mg/kg/day) mice were significantly higher than NFD mice (*p<0.05). At Day 31, mean non-fasted blood glucose values of HFD remained significantly elevated (*p<0.05); whereas, NFD and HFD-Tx (200 mg/kg/day) mice were not significantly different. Data are represented as means±SEM. FIG. 8 shows the fasted blood glucose values on the final treatment day (day 42). In HFD mice, mean fasted blood glucose values were significantly higher as compared to NFD and HFD-Tx (200 mg/kg/day) mice (*p<0.05). Data are represented as means±SEM.

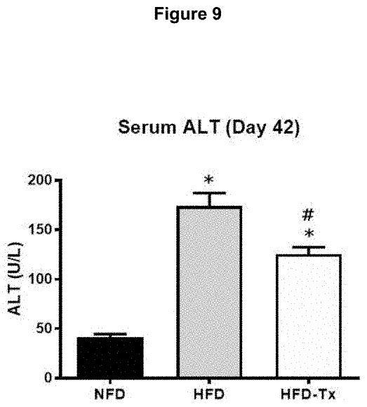

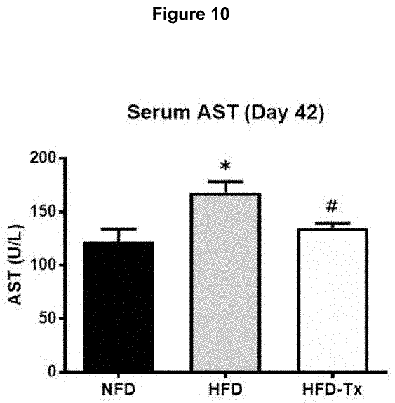

FIG. 9 shows the mean serum alanine aminotransferase (ALT) values on the final study day (day 42). The serum ALT values of HFD and HFD-Tx (200 mg/kg/day) mice were significantly higher compared to NFD mice (*p<0.05). Among high-fat fed mice, serum ALT values in HFD-Tx (200 mg/kg/day) mice were significantly lower than in HFD mice (# p<0.05). Data are represented as means±SEM. FIG. 10 shows the mean serum aspartate aminotransferase (AST) values on the final study day (day 42). The serum AST values of HFD mice were significantly higher compared to NFD and HFD-Tx (200 mg/kg/day) mice (*p<0.05). Among high-fat fed mice, serum AST values in HFD-Tx (200 mg/kg/day) mice were significantly lower than in HFD mice (# p<0.05). Data are represented as means±SEM.

While not wishing to be bound by any particular theory, the inventors believe that the combination of Nigella sativa, Kaempferia parviflora, and Rosa canina may be useful in methods of weight loss, including synergistic interactions enhancing some of the effects, while the relative proportions of the components in the composition may provide some degree of interference of some effects. Furthermore, without being bound by theory, the inventors believe that the specific compositions disclosed herein as used and tested may be useful as a supportive composition for blood glucose control and liver health/protection when used solely for that purpose or for general health, when used in combination with one or more other ingredients for weight loss, or in conjunction with ingredients for other protocols such as, for example, weight gain, weight maintenance, muscle gain, cardiovascular training, or endurance training.

More specifically, the inventors believe that when combined with ingredients used for weight loss, the composition of Nigella sativa, Kaempferia parviflora, and Rosa canina may enhance weight loss, or alternatively, not interfere with weight loss, while providing the benefits of controlling blood glucose levels and/or supporting liver health by offering liver protection. Such weight loss ingredients are known to those skilled in the art and include but are not limited to: caffeine, green tea extract, green coffee bean extract, bitter orange (synephrine), conjugated linoleic acid, L-carnitine, African mango, and hydroxycitric acid.

The compositions of the present invention may be administered by any suitable means, including orally, sublingually, intravenously and topically. The preferred dosage forms are oral, and include ingestion as a solid, pill, tablet, liquid tablet, caplet or capsule, in a powder form or powdered beverage mix, suspended in water or other liquid, or as a dietary gel, and may be taken by itself or incorporated into compositions that further comprise other ingredients, such as, but not limited to, weight loss ingredients, as described above, additional active ingredients and/or inactive ingredients, including solvents, diluents, suspension aids, thickening or emulsifying agents, sweeteners, flavorings, preservatives, solid binders, lubricants and the like, as suited to the particular dosage form desired.

The above-described embodiments are intended to be examples of the present invention and alterations and modifications may be effected thereto, by those of skill in the art. Such alterations and modifications are contemplated and do not take the compositions described outside of the scope of the invention. The scope of the claims should not be limited by the preferred embodiments set forth in the examples, but should be given the broadest interpretation consistent with the description as a whole.

All publications which are cited herein are hereby specifically incorporated by reference into the disclosure for the teachings for which they are cited.

What is claimed is:

1. A method for controlling blood glucose levels comprising administering to a subject in need thereof a composition comprising a Nigella sativa extract, a Kaempferia parviflora extract, and a Rosa canina extract.

2. The method of claim 1, wherein the ratio of Nigella sativa to Kaempferia parviflora to Rosa canina is 9:1:1.

3. The method of claim 2, wherein the total amount of the composition administered is 1.1 g/day.

4. The method of claim 1, wherein the composition is administered to an individual desiring to lose weight.

5. The method of claim 4, wherein the composition is part of a composition further comprising additional ingredients to aid in weight loss.

6. The method of claim 5, wherein the ratio of Nigella sativa to Kaempferia parviflora to Rosa canina in the composition is 9:1:1.

7. The method of claim 6, wherein the total amount of the Nigella sativa, the Kaempferia parviflora, and the Rosa canina in the composition administered is 1.1 g/day.

8. The method of 2, wherein the Nigella sativa extract is a water seed extract.

9. The method of claim 2, wherein the Kaempferia parviflora extract is an ethanol/water root extract.

10. The method of claim 2, wherein the Rosa canina extract is an ethanol/water fruit extract.

11. A method for liver protection comprising administering to a subject in need thereof a composition comprising a Nigella sativa extract, a Kaempferia parviflora extract, and a Rosa canina extract.

12. The method of claim 6, wherein the ratio of Nigella sativa to Kaempferia parviflora to Rosa canina is 9:1:1.

13. The method of claim 7, wherein the total amount of the composition administered is 1.1 g/day.

14. A method for controlling blood glucose levels and liver protection comprising administering to a subject in need thereof a composition comprising a Nigella sativa extract, a Kaempferia parviflora extract, and a Rosa canina extract.

15. The method of claim 1, wherein the ratio of Nigella sativa to Kaempferia parviflora to Rosa canina is 9:1:1.

16. The method of claim 2, wherein the total amount of the composition administered is 1.1 g/day.

17. A composition for controlling blood glucose levels and liver protection comprising Nigella sativa extract, a Kaempferia parviflora extract, and a Rosa canina extract.

18. The composition of claim 17, wherein the ratio of Nigella sativa to Kaempferia parviflora to Rosa canina is 9:1:1.

19. The method of claim 17, wherein the total amount of the composition administered is 1.1 g/day

20. The composition of claim 17, wherein the wherein the Nigella sativa extract is a water seed extract, the Kaempferia parviflora extract is an ethanol/water root extract, the Rosa canina extract is an ethanol/water fruit extract.
